(19) United States
(12) Patent Application Publication
(10) Pub. No.: US 2025/0319224 A1
(43) Pub. Date: Oct. 16, 2025

(54) VOLATILE COMPOSITION DISPENSER WITH REPLACEABLE CONTAINER

(71) Applicant: The Procter & Gamble Company, Cincinnati, OH (US)

(72) Inventors: William Michael Cannon, West Harrison, IN (US); Rance Kyndall PRITCHARD, Cincinnati, OH (US); David TURNER, Cincinnati, OH (US)

(21) Appl. No.: 19/172,771

(22) Filed: Apr. 8, 2025

Related U.S. Application Data
(60) Provisional application No. 63/633,118, filed on Apr. 12, 2024.
(60) Provisional application No. 63/733,482, filed on Dec. 13, 2024.

(51) Int. Cl. A61L 9/03

(52) U.S. Cl. CPC A61L 9/032; A61L 2209/133; A61L 2209/135; A61L 2209/15

(57) ABSTRACT

A volatile composition dispenser includes a housing having a top portion and a replaceable volatile composition container that releasably engages with the housing. When the replaceable volatile composition container is releasably engaged with the housing, the replaceable volatile composition container is positioned partially within the top portion of the housing. The replaceable volatile composition container includes a sidewall, and the sidewall includes a first gripping portion. At least a portion of the first gripping portion is positioned at least about 5 mm below the top portion of the housing.

### TECHNOLOGICAL FIELD

The present disclosure relates to volatile composition dispensers with replaceable volatile composition containers.

### BACKGROUND

Volatile composition dispensers exist for delivering various volatile compositions, such as freshening compositions, into the air. Such volatile composition dispensers may, for example, take the form of a wick-based electrical dispenser having one or more heaters to assist with volatizing the volatile composition into the air. Consumers desire for easy replacement of the replaceable volatile composition containers within the volatile composition dispensers. However, conventional replaceable volatile composition containers may be difficult to grip when inside the volatile composition dispenser. Thus, there remains a need to deliver replaceable volatile composition containers that provide easier gripping.

### SUMMARY

According to an example of the present disclosure, a volatile composition dispenser includes a housing having a top portion and a replaceable volatile composition container that releasably engages with the housing. When the replaceable volatile composition container is releasably engaged with the housing, the replaceable volatile composition container is positioned partially within the housing. The replaceable volatile composition container includes a sidewall, and the sidewall includes a first gripping portion. At least a portion of the first gripping portion is positioned at least about 5 mm below the top portion of the housing.

According to another example of the present disclosure, a volatile composition dispenser includes a housing including a top portion; and a replaceable volatile composition container that releasably engages with the housing, and when the replaceable volatile composition container is releasably engaged with the housing, the replaceable volatile composition container is positioned partially within the housing. The replaceable volatile composition container includes a sidewall, and the sidewall includes a first gripping portion. At least a portion of the first gripping portion is positioned at least about 5 mm below the top portion of the housing, and the volatile composition dispenser comprises an evaporative assistance element comprising a heater or a fan. The evaporative assistance element is electrically connected to an outlet connector, and the outlet connector is electrically connected to the volatile composition dispenser through a cord.

### BRIEF DESCRIPTION OF THE DRAWINGS

The following detailed description of specific examples of the present disclosure can be best understood when read in conjunction with the following drawings, where like structure is indicated with like reference numerals and wherein:

FIG. 1 is a perspective view of volatile composition dispenser, in accordance with examples of the present disclosure.

FIG. 2 is a profile view of a volatile composition dispenser, in accordance with examples of the present disclosure.

FIG. 3 is a profile view of a volatile composition dispenser, in accordance with examples of the present disclosure.

FIG. 4 is a profile view of a volatile composition dispenser, in accordance with examples of the present disclosure.

FIG. 5 is a top-down view of a replaceable volatile composition container, in accordance with examples of the present disclosure.

FIG. 6 is a cut-away view of the replaceable volatile composition container of FIG. 5, in accordance with examples of the present disclosure.

FIG. 7 is a chart showing the heating activity over time for two heaters, in accordance with examples of the present disclosure.

### DETAILED DESCRIPTION

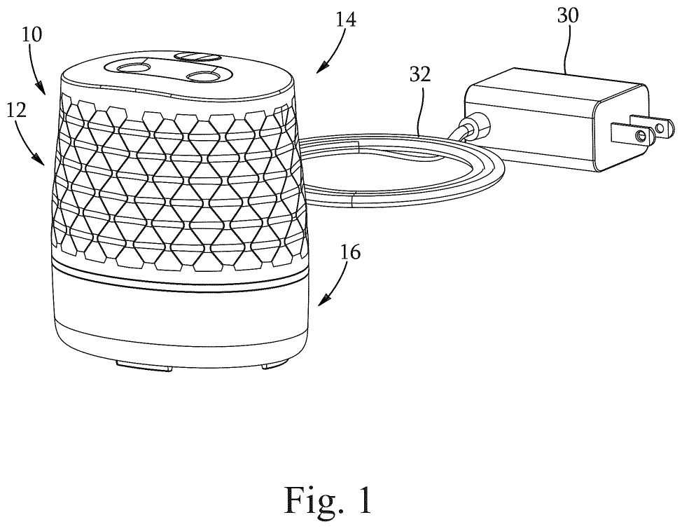

Referring initially to FIGS. 1-4, a volatile composition dispenser 10 may include a housing 12 having a top portion 14 and a replaceable volatile composition container 20. It is contemplated that the volatile composition dispenser 10 may be configured for use in a variety of applications to deliver the volatile composition to the air and/or ultimately to a surface. The volatile composition dispenser 10 may be configured in various ways. For example, the volatile composition dispenser 10 may be configured as an electrical wall plug volatile composition dispenser. The volatile composition dispenser 10 may include a visual or tactile feature 11, such as a light or a button.

As stated previously, the volatile composition dispenser 10 may include a housing 12 and a replaceable volatile composition container 20. The housing 12 may include a top portion 14. The top portion 14 may have a bottom end 15. In examples, the housing 12 may include a removable bottom portion 16, which may have a top end 17. The top portion 14 and the bottom portion 16 may be removably coupled. For example, the bottom end 15 of the top portion 14 may be positioned adjacent to the top end 17 of the bottom portion 16.

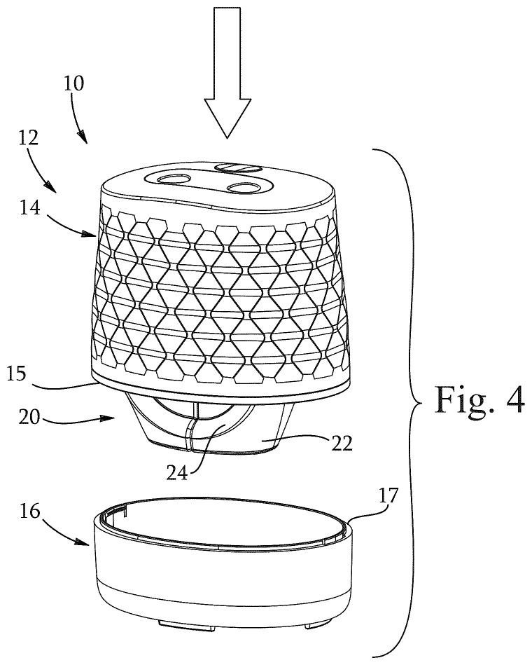

The replaceable volatile composition container 20 may releasably engage with the housing 12, and when the replaceable volatile composition container 20 is releasably engaged with the housing 12, the replaceable volatile composition container 20 may be positioned partially within the housing 12 (as depicted in FIG. 4).

The replaceable volatile composition container 20 may further include a sidewall 22. The sidewall 22 may include a first gripping portion 24. The first gripping portion 24 may be a protrusion (as shown), or may be any other form that allows for case of gripping, such as a textured portion or an indent (not shown). It is contemplated that in examples where the first gripping portion 24 is a textured portion, the textured portion may have a coefficient of friction that is greater than the portion of the sidewall 22 that is not textured, allowing for easier gripping as compared to the portion of the sidewall 22 that is not textured. In examples, the first gripping portion 24 may be made of a different material than the sidewall 22, such as rubber. It is contemplated that in examples where the first gripping portion 24 may be made of rubber, the first gripping portion 24 may be easier to grip as compared to the sidewall 22. The first gripping portion 24 may be any shape suitable for gripping. In examples, the first gripping portion 24 may be half-circle shaped or half-ovoid shaped. In examples, the first gripping portion 24 may include a half-circle shaped or a half-ovoid shaped protrusion.

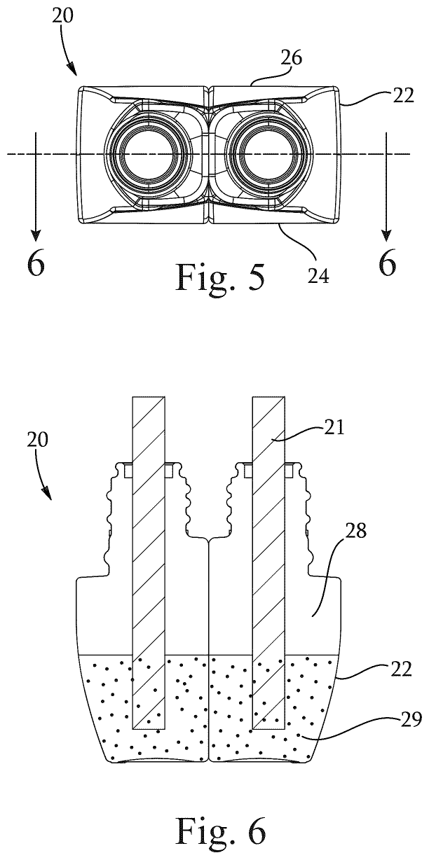

Referring now to FIG. 5, in examples, the sidewall 22 of the replaceable volatile composition container 20 further includes a second gripping portion 26 positioned opposite the first gripping portion 24. The second gripping portion 26 may be any of the examples of the first gripping portion 24 as described previously.

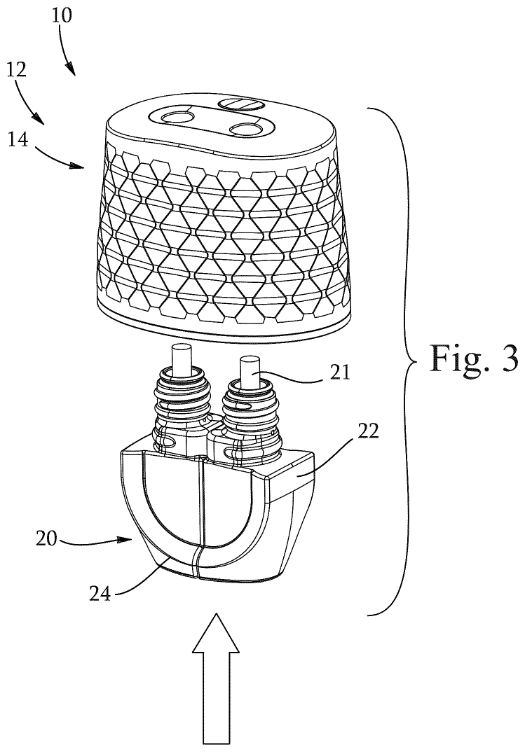

Referring to FIGS. 3-5, in examples, at least a portion of the first gripping portion 24, the second gripping portion 26, or both, is positioned at least about 5 millimeters (mm) below the top portion 14 of the housing 12 (e.g., below the bottom end 15 of the top portion 14). In examples, the first gripping portion 24, the second gripping portion 26, or both, may be positioned at least about 5 millimeter (“mm”), at least about 8 mm, at least about 10 mm, from about 5 to about 50 mm, from about 5 to about 30 mm, from about 5 to about 20 mm, from about 5 to about 15 mm, from about 5 to about 12 mm, from about 5 to about 10 mm, from about 5 to about 8 mm, from about 8 to about 20 mm, from about 8 to about 15 mm, from about 8 to about 12 mm, from about 8 to about 10 mm, from about 10 to about 20 mm, from about 10 to about 15 mm, from about 10 to about 12 mm, or any values within the foregoing ranges or any ranges created thereby, below the top portion 14 of the housing 12. Positioning the first gripping portion 24, the second gripping portion 26, or both, at least 5 mm below the bottom end 15 of the top portion 14 of the housing 12 allows a user to fit their finger, such as a thumb, between the top portion 14 of the housing 12 and the first gripping portion 24, the second gripping portion 26, or both, and easily to exert downward pressure to remove the replaceable volatile composition container 20 from the housing 12.

In examples, at least a portion of the first gripping portion 24, the second gripping portion 26, or both, protrudes at least about 0.5 mm, at least about 1 mm, from about 0.5 to about 5 mm, from about 0.5 to about 3 mm, from about 0.5 to about 2 mm, from about 0.5 to about 1.5 mm, from about 0.5 to about 1 mm, from about 1 to about 5 mm, from about 1 to about 3 mm, from about 1 to about 2 mm, from about 1 to about 1.5 mm, or any values within the foregoing ranges or any ranges created thereby, from the sidewall 22. In examples, the term “from the sidewall” refers to from an upper portion of the sidewall where the protrusion is not present. A protrusion of at least about 0.5 mm allows a user to rest a finger, such as a thumb, on the first gripping portion 24, the second gripping portion 26, or both, and easily to exert downward pressure to remove the replaceable volatile composition container 20 from the housing 12.

Referring now to FIG. 6, in examples, the sidewall 22 of the replaceable volatile composition container 20 at least partially encompasses a volatile composition reservoir 28. In examples, the volatile composition reservoir 28 may include a volatile composition 29. The sidewall 22 can be made of any suitable material. Suitable materials for construction of the sidewall include, but are not limited to, glass and plastic. The sidewall 22 may be constructed of any type of container that is suitable for holding volatile compositions.

The sidewall 22 may form more than one volatile composition reservoir 28. In examples, the sidewall 22 may form one (not shown), two (as shown in FIG. 6), three (not shown), four (not shown), or five (not shown) volatile composition reservoirs 28. It is also possible for each volatile composition reservoir 28 to have different types of volatile composition.

The term “volatile compositions” as used herein, refers to a material that comprises a vaporizable material. The term “volatile compositions,” thus includes (but is not limited to) compositions that are comprised entirely of a single volatile material. The terms “volatile materials,” “aroma,” “fragrance,” and “scents,” as used herein, include, but are not limited to pleasant or savory smells, and, thus, also encompass materials that function as insecticides, air fresheners, deodorants, aromacology, aromatherapy, insecticides, or any other material that acts to condition, modify, or otherwise charge the atmosphere or to modify the environment. It should be understood that certain volatile compositions including, but not limited to perfumes, aromatic materials, and scented materials, will often comprise one or more volatile materials (which may form a unique and/or discrete unit comprised of a collection of volatile materials). It should be understood that the term “volatile composition” refers to compositions that have at least one volatile component, and it is not necessary for all of the component materials of the volatile composition to be volatile. The volatile compositions described herein may, thus, also have non-volatile components. It should also be understood that when the volatile compositions are described herein as being “emitted,” this refers to the volatilization of the volatile components thereof and does not require that the non-volatile components thereof be emitted. The volatile compositions of interest herein can be in any suitable form including, but not limited to, solids, liquids, gels, encapsulates, and combinations thereof.

Referring still to FIG. 6, in examples, the volatile composition dispenser 10 may further include a delivery engine that may be used to transport the volatile composition to an evaporative surface 21. The delivery engine may include an evaporative surface 21. The evaporative surface 21 may be in fluid communication with the volatile composition reservoir 28. In such a configuration, the delivery engine may be placed next to one or more evaporative assistance elements, such as a heater or fan to volatilize the volatile composition into the air. The evaporative assistance elements may surround, or at least partially surround, the evaporative surface 21.

Referring now to FIGS. 1 and 6, in examples, the evaporative assistance element may be electrically connected to an outlet connector 30. The outlet connector 30 may be electrically connected to the volatile composition dispenser 10 through a cord 32 through any means known in the art. In examples, the cord 32 may have a length of at least about 10 centimeters (cm), at least about 20 cm, at least about 30 cm, at least about 50 cm, from about 10 to about 300 cm, from about 10 to about 200 cm, from about 10 to about 150 cm, from about 10 to about 130 cm, from about 10 to about 100 cm, from about 10 to about 50 cm, from about 90 to about 300 cm, from about 90 to about 200 cm, from about 90 to about 150 cm, from about 90 to about 130 cm, from about 90 to about 100 cm, from about 100 to about 300 cm, from about 100 to about 200 cm, from about 100 to about 150 cm, from about 100 to about 130 cm, from about 130 to about 300 cm, from about 130 to about 200 cm, from about 130 to about 150 cm, from about 150 to about 300 cm, from about 150 to about 200 cm, or any values within the foregoing ranges or any ranges created thereby.

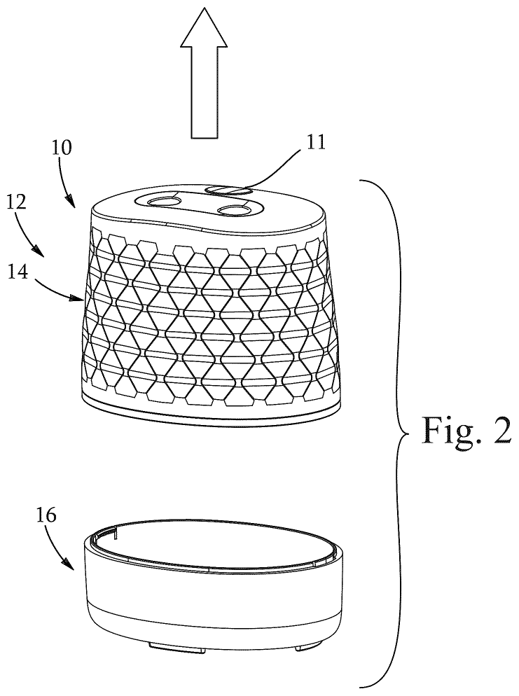

The user may insert the volatile composition container 20 into the volatile composition dispenser 10 by first separating the top portion 14 from the bottom portion 16, as shown in FIG. 2. The volatile composition container 20 may be removably coupled to the top portion 14, as shown in FIG. 3. Once the volatile composition container 20 is positioned in the top portion 14, the top portion 14 may be removably coupled to the bottom portion 16.

As described previously, one or more evaporative assistance elements, such as a heater or fan, may be used to volatilize the volatile composition into the air. The evaporative assistance element(s) may be heated according to a heating cycle. The heating cycle may involve a steady or variable level of energy. The energy may be continuous or discontinuous. The heating cycle may include periods of increased energy (“energy boost”), decreased energy, and/or maintained energy to provide improved consumer acceptance of a volatile composition dispenser over commercially available volatile composition dispensers.

The volatile composition dispenser 10 may include a button (e.g., feature 11) for adjusting the energy supplied to the evaporative assistance element(s). For example, the volatile composition dispenser 10 may include an energy boost button. An energy boost at various extended emission periods over the life of the volatile composition in the reservoir can increase noticeability of the volatile composition over the use of the dispenser by maintaining or increasing the evaporation rate of the volatile composition from the evaporative surface 21 over time.

When the boost phase is activated (e.g., when the boost button is pressed), the current heating cycle is interrupted. The boost phase may operate for a defined period of time before the volatile composition dispenser 10 returns to the previous heating cycle. The energy boost may have a discrete emission period(s) of about 1 minute to about 90 minutes, about 10 minutes to about 60 minutes, or about 30 minutes to about 50 minutes. For example, the energy boost may have a discrete emission period of about 30 minutes, about 40 minutes, or about 50 minutes.

The boost phase may involve activating one or more heaters or fans. In an embodiment, the boost phase involves two heaters operating in series with a first heater operating for a first period followed by the second heater operating for a second time period. The first and second heaters may be configured to volatilize the volatile composition in the same reservoir 28 or different reservoirs 28. The first time period and the second time period may be the same or may be different. For example, the first time period and/or the second time period may be about 10 minutes, about 15 minutes, about 20 minutes, or about 30 minutes. The first time period and/or the second time period may be at least as long as required for the evaporative surface 21 (e.g., a wick) to reach a desired temperature.

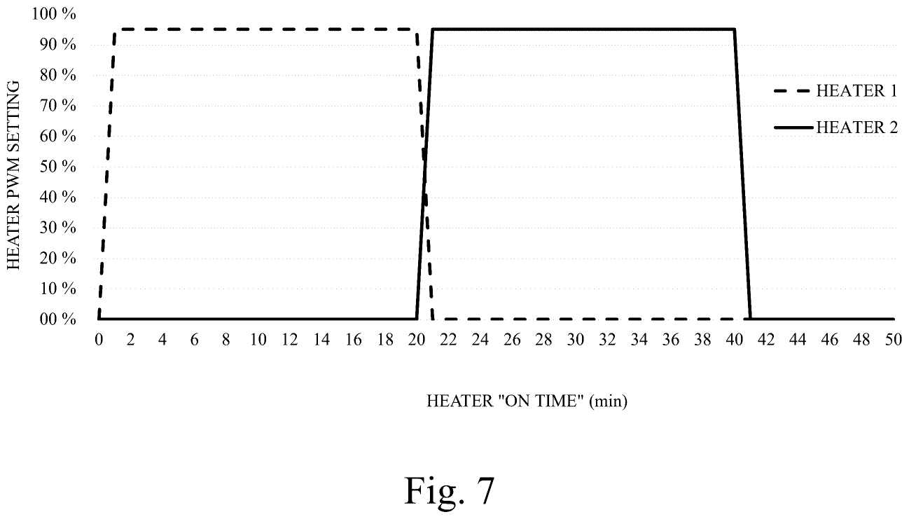

There may be a time lag between activating the first and/or second heater and reaching a steady state. In some examples, the second heater may be activated while the first heater is deactivating. As shown in FIG. 7, a first heater may be operated for about 20 minutes followed by the second heater operating for about 20 minutes. This may provide a steady level of energy over the boost phase while using two heaters. As described previously, the first and second heaters may be configured to volatilize a volatile composition in different reservoirs 28. Using the first and second heaters in a series may enhance noticeability by giving the consumer two distinct periods of peaks and valleys of fragrances release. The boost phase as shown in FIG. 7, for example, results in a first period with a peak upon heating of the first evaporative surface 21, a second period of a brief depression or valley in the boost cycle during the heating of a second evaporative surface 21 where there is an overlap (e.g., 10 min) while the first evaporative surface 21 is cooling down and the second evaporative surface 21 is heating up, and a third period with a peak heating period for this second evaporative surface 21. The third period is followed by a cool down period where the program returns to the previous operation. The power level of the first and/or second heater in the previous operation is lower than any heater power level during the boost phase.

It may be desirable to have a corded volatile composition dispenser that also has an casily removable refill. A volatile composition dispenser that has a cord may result in improved performance within the environment, as shown in the Examples. Specifically, consumers noted that a corded volatile composition dispenser exhibited increased scent consistency in the room over time as compared to a plug-in volatile composition dispenser (cordless), increased noticeability when entering the room or home, increased home and room fill, increased odor elimination, and increased scent overall. Without being bound by theory, this is contemplated to be due to the ability to place a corded volatile composition dispenser on a table and therefore further away from a wall.

This spacing of the dispenser away from the wall utilizes air movement within a room that may not be as readily available in locations close to/on a wall. The air movement allows scent to more easily be spread with in a space. Additionally, a corded volatile composition dispenser also having an easily removable refill combines ease of use (as described previously) with the improved performance of a corded volatile composition dispenser. Better performing volatile composition dispensers that are easier to use are in continued demand.

In examples, instead of evaporating the volatile composition from an evaporative surface 21 of the delivery engine, the delivery engine may transport the volatile composition to a separate evaporative surface (not shown). The evaporative surface may be configured as a porous or semi-porous substrate, a bowl or plate, including a plastic, glass, or metal bowl or plate, and combinations thereof.

The evaporative surface 21 may be configured in various ways. For example, the evaporative surface 21 may be in the form of a wick, membrane, gel, wax, porous or semi-porous substrate, including a felt pad, or a combination thereof. In a volatile composition dispenser 40 comprising more than one delivery engine associated with the same or different reservoirs (not shown), the delivery engines may be the same or may be different.

If the volatile composition dispenser utilizes a wick as the evaporative surface 21, the wick may be configured to have various different shapes and sizes. For example, the wick may have a cylindrical or an elongate cube shape. The wick may be defined by a length and a diameter or width, depending on the shape. The wick may have various lengths. For example, the length of the wick may be in the range of about 1 mm to about 100 mm, or from about 5 mm to about 75 mm, or from about 10 mm to about 50 mm. The wick may have various diameters or widths. For example, diameter or width of the wick may be at least 1 mm, or at least 2 mm, or at least 3 mm, or at least 4 mm. A wick may exhibit a density. The wick density may be in the range of about 0.100 grams/cm³ (“g/cc”) to about 1.0 g/cc.

A wick may comprise a porous or semi-porous substrate. The wick may be composed of various materials and methods of construction, including, but not limited to, bundled fibers which are compressed and/or formed into various shapes via overwrap (such as a non-woven sheet over-wrap) or made of sintered plastics such as PE, HDPE, or other polyolefins. The wick may be made from a plastic material such as polyethylene or a polyethylene blend.

### Examples

Consumer tests were run where 80 consumers were given a plug-in product and a corded product according to the present disclosure to use over 6 weeks. The below table show the consumer preferences out of 100.

TABLE 1

| | Plug-in | Corded product according to |
|---|---|---|
| | product | the present disclosure |
| Overall Rating | 56 | 70 |
| Scent Overall | 73 | 82 |
| Scent is Authentic | 67 | 76 |
| Scent amount in room is | 48 | 61 |
| consistent over time | | |
| Room Fill | 56 | 71 |
| House Fill | 31 | 49 |
| Noticeability when entering | 63 | 73 |
| room | | |
| Noticeability when entering | 56 | 65 |
| home | | |
| Travel throughout your home | 35 | 51 |
| Odor Elimination | 59 | 74 |

There were improvements in survey responses on room & house fill and scent travel throughout the home. Many panelists qualitatively mentioned that they could smell the scent in new areas of their home. This confirmed the hypothesis that this device would be able to reach more areas of the home because it could be plugged in away from the wall/floor and it was less likely to be blocked by furniture. Additionally, many panelists mentioned that they would use the boost option when there was a situational odor (e.g., cooking, pet, trash, diapers, etc.).

Additionally, panelists were asked to evaluate the intensity of the device from 5 ft and 10 ft distances and saw significant improvements in intensity ratings of the new device compared to the control plugged in on the wall. Panelists noted improvements on odor reduction with the corded device in accordance with the present disclosure.

Most panelists placed the corded device in accordance with the present disclosure on coffee or side tables ranged from 12 to 30 inches in height. Additionally, the corded device in accordance with the present disclosure has a 6-foot cord allowing it to be moved further away from the wall. The non-corded plug-ins today have to be placed at the outlet on the wall, and most standard outlets are about 1 ft from the floor. The general area where the corded devices in accordance with the present disclosure were placed was at least 1 foot in the vertical direction and greater than 6 inches from the wall in the horizontal direction.

While devices, systems, compositions, or methods are described herein in terms of “comprising” various components or steps, the devices, systems, compositions, or methods can also “consist essentially of” or “consist of” the various components or steps, unless stated otherwise.

As used herein, the word “or” when used as a connector of two or more elements is meant to include the elements individually and in combination. For example, “X or Y” means X or Y or both.

As used herein, the articles “a” and “an” are understood to mean one or more of the material that is claimed or described, for example, “a volatile composition” or “a connector”, unless stated otherwise.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any example disclosed or claimed herein or that it alone, or in any combination with any other reference or references, teaches, suggests, or discloses any such example. Further, to the extent that any meaning or definition of a term in this document conflicts with any meaning or definition of the same term in a document incorporated by reference, the meaning or definition assigned to that term in this document shall govern.

While particular examples of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this disclosure.

What is claimed is:

1. A volatile composition dispenser comprising:
a housing comprising a top portion; and
a replaceable volatile composition container that releasably engages with the housing, and when the replaceable volatile composition container is releasably engaged with the housing, the replaceable volatile composition container is positioned partially within the top portion of the housing, wherein:
the replaceable volatile composition container comprises a sidewall, and
the sidewall comprises a first gripping portion, wherein at least a portion of the first gripping portion is positioned at least about 5 mm below the top portion of the housing.

2. The volatile composition dispenser of claim 1, wherein at least a portion of the first gripping portion is positioned at least about 8 mm below the top portion of the housing.

3. The volatile composition dispenser of claim 1, wherein at least a portion of the first gripping portion is positioned at least about 10 mm below the top portion of the housing.

4. The volatile composition dispenser of claim 1, wherein at least a portion of the first gripping portion protrudes at least about 0.5 mm from the sidewall.

5. The volatile composition dispenser of claim 1, wherein at least a portion of the first gripping portion is a protrusion that extends at least about 1 mm from the sidewall.

6. The volatile composition dispenser of claim 1, wherein the first gripping portion comprises a half-circle shaped or half-ovoid shaped protrusion.

7. The volatile composition dispenser of claim 1, wherein the sidewall further comprises a second gripping portion that comprises a second protrusion positioned opposite the first gripping portion.

8. The volatile composition dispenser of claim 7, wherein the second gripping portion is positioned at least about 8 mm below the top portion of the housing, preferably at least about 10 mm below the top portion of the housing.

9. The volatile composition dispenser of claim 7, wherein the second gripping portion protrudes at least about 0.5 mm from the sidewall, preferably at least about 1 mm from the sidewall.

10. The volatile composition dispenser of claim 7, wherein:
the first gripping portion comprises a first half-circle shaped or half-ovoid shaped protrusion; and
the second gripping portion further comprises a second half-circle shaped or half-ovoid shaped protrusion opposite the first half-circle shaped or half-ovoid shaped protrusion.

11. The volatile composition dispenser of claim 1, wherein the housing further comprises a removable bottom portion.

12. The volatile composition dispenser of claim 1, wherein the sidewall at least partially encompasses a volatile composition reservoir.

13. The volatile composition dispenser of claim 12, wherein the volatile composition reservoir comprises a volatile composition.

14. The volatile composition dispenser of claim 12, wherein the volatile composition reservoir comprises two separate reservoirs.

15. The volatile composition dispenser of claim 14, wherein the two separate reservoirs each comprise a volatile composition.

16. The volatile composition dispenser of claim 12, wherein the replaceable volatile composition container comprises an evaporative surface in fluid communication with the volatile composition reservoir.

17. The volatile composition dispenser of claim 1, wherein the volatile composition dispenser further comprises an evaporative assistance element comprising a heater or a fan.

18. The volatile composition dispenser of claim 17, wherein the housing further comprises a button operably configured to activate the heater or the fan.

19. The volatile composition dispenser of claim 18, wherein the evaporative assistance element comprises a first heater and a second heater, and the button is operably configured to activate the first heater at a first time and, at a second time later than the first time, deactivate the first heater and activate the second heater.

20. A volatile composition dispenser comprising:
a housing comprising a top portion; and
a replaceable volatile composition container that releasably engages with the housing, and when the replaceable volatile composition container is releasably engaged with the housing, the replaceable volatile composition container is positioned partially within the top portion of the housing, wherein:
the replaceable volatile composition container comprises a sidewall,
the sidewall comprises a first gripping portion, wherein at least a portion of the first gripping portion is positioned at least about 5 mm below the top portion of the housing,
the volatile composition dispenser comprises an evaporative assistance element comprising a heater or a fan,
the evaporative assistance element is electrically connected to an outlet connector; and
the outlet connector is electrically connected to the volatile composition dispenser through a cord.
